SEQUENCE LISTING (13 sequences)

SEQ ID NO: 1 - protein, 7 aa, Artificial Sequence
  Description of Artificial Sequence Synthetic peptide
CHLDIIW

SEQ ID NO: 2 - protein, 24 aa, Artificial Sequence
  Description of Artificial Sequence Synthetic peptide
CGYWGDIMGEWGDEIFGEIAGFLG

SEQ ID NO: 3 - protein, 8 aa, Artificial Sequence
  Description of Artificial Sequence Synthetic peptide
GRVYIHPT

SEQ ID NO: 4 - protein, 8 aa, Artificial Sequence
  Description of Artificial Sequence Synthetic peptide
GRVYVHPA

SEQ ID NO: 5 - protein, 19 aa, Artificial Sequence
  Description of Artificial Sequence Synthetic peptide
ARYYSALRHYINLITRQRY

SEQ ID NO: 6 - protein, 16 aa, Artificial Sequence
  Description of Artificial Sequence Synthetic peptide
EEEEEMAVVPQGLFRG

SEQ ID NO: 7 - protein, 29 aa, Artificial Sequence
  Description of Artificial Sequence Synthetic peptide
YADAIFTSNYRKVLGQLSARKLLQDIMSR

SEQ ID NO: 8 - protein, 10 aa, Artificial Sequence
  Description of Artificial Sequence Synthetic peptide
XHWSYGLRPG

SEQ ID NO: 9 - protein, 13 aa, Artificial Sequence
  Description of Artificial Sequence Synthetic peptide
CNAEVYVVPWEKK

SEQ ID NO: 10 - protein, 27 aa, Artificial Sequence
  Description of Artificial Sequence Synthetic peptide
LDCNAEVYVVPWEKKIYPTVNCQPLGM

SEQ ID NO: 11 - protein, 6 aa, Artificial Sequence
  Description of Artificial Sequence Synthetic peptide
HLDIIW

SEQ ID NO: 12 - protein, 5 aa, Artificial Sequence
  Description of Artificial Sequence Synthetic peptide
LDIIW

SEQ ID NO: 13 - protein, 8 aa, Artificial Sequence
  Description of Artificial Sequence Synthetic peptide
EGVYVHPV

(19) United States
(12) Patent Application Publication
(10) Pub. No.: US 2009/0220587 A1
(43) Pub. Date: Sep. 3, 2009

(54) Liposomal drug delivery constructs targeted by lipid-conjugated peptide ligands

(75) Inventors: Nahum Allon, Ness-Ziona (IL); Ashima Saxena, Silver Spring, MD (US); Carolyn Chambers, Silver Spring, MD (US); Bhupendra P. Doctor, Silver Spring, MD (US)

Correspondence Address: DRINKER BIDDLE & REATH (DC), 1500 K STREET, N.W., SUITE 1100, WASHINGTON, DC, 20005-1209, US

(73) Assignee: UNITED STATE ARMY, FREDERICK, MD (US)

(21) Appl. No.: 12/379,096

(22) Filed: Feb. 12, 2009

Related U.S. Application Data
(63) Continuation-in-part of application No. 11/339,404, filed on Jan. 25, 2006.
(60) Provisional application No. 60/649,034, filed on Feb. 1, 2005.

(51) Int. Cl. A61K 9/127; C07K 2/00; C07K 7/06; C07K 7/08; C07K 14/00; A61K 31/7052; A61P 43/00

(52) U.S. Cl. 424/450; 530/300; 530/330; 530/329; 530/328; 530/327; 530/326; 530/325; 530/324; 530/323; 514/44 R; 514/44 A; 264/4.6

(57) ABSTRACT

A receptor ligand is conjugated to a lipid-soluble moiety, thereby allowing effective delivery of the peptide ligand to a target cell in the form of a liposome complex. The ligand brings the liposome in proximity to the target cell, facilitating the fusion of the liposome with an endosome of the cellular target to release the payload into the cytoplasm. The liposome itself packages drugs or other therapeutics that are delivered to the interior of the cell upon fusion of the liposome.

### CROSS REFERENCE TO RELATED APPLICATIONS

The present application is a continuation-in-part of U.S. application Ser. No. 11/339,404, filed Jan. 25, 2006, which claims the benefit of priority under 35 U.S.C. § 119(e) to U.S. Provisional Application No. 60/649,034, filed Feb. 1, 2005, each of which is incorporated by reference herein in its entirety.

### SEQUENCE LISTING

A Sequence Listing, comprising SEQ ID NOS: 1-13, follows this specification and is incorporated herein by reference in its entirety.

### FEDERAL SUPPORT

This invention was made by employees of the United States Army. The government has rights in the invention.

### FIELD OF THE INVENTION

Compositions and methods related to liposomal drug delivery constructs targeted by lipid-conjugated peptides, which are ligands for cell surface receptors.

### BACKGROUND

Targeting drugs, oligonucleotides, and genes to a specific tissue, and to cells have been fundamental goals of the pharmaceutical industry. Until recently, these have been elusive problems.

One of the fundamental properties of living cells is their ability to sense and respond to their environment. This is accomplished by a specific set of receptors on the cell surface. Identifying the specific binding proteins and defining their specificity are big strides towards resolving the problem of specific tissue targeting.

Encapsulating drugs in liposomes and other microcapsules opens new lines of research. Encapsulation can deliver a large number of drug molecules that are targeted towards a specific tissue, by protecting them from degradation by enzymes. Accomplishing this goal may reduce or even eliminate side effects by reducing the amount of drug needed and increasing its effectiveness due to its accumulation in the target tissue. Since most of the targeting peptides are water-soluble and the phospholipids are oil soluble, specific linkers and several steps are required for binding these two molecules. The situation gets more complicated when the conjugation of several peptides at different concentrations is required for the delivery of a gene. Further, conjugating targeting peptides to drugs often raises technical problems and in most cases affects the activity of the drug.

### SUMMARY

A receptor ligand is conjugated to a lipid-soluble moiety, thereby allowing effective delivery of the conjugated peptide ligand to a target cell in the form of a liposome complex. The ligand brings the liposome in proximity to the target cell, facilitating the fusion of the liposome with the plasma membrane of the cellular target. The liposome itself packages drugs or other therapeutics that are delivered to the interior of the cell upon fusion of the liposome. Among the many advantages of the present liposome complexes, they may be rapidly removed from circulation by co-internalization with the peptide ligand's cognate receptor, thereby avoiding internalization by non-target cells.

Accordingly, one aspect of the present disclosure provides a liposome complex comprising:

(i) a conjugated peptide ligand of formula (I):

peptide ligand-X-fatty acid (I); and

(ii) a liposome comprising about 10-40 mole percent phosphatidyl serine compared to total lipids, and wherein the liposome is capable of fusing with a cellular plasma membrane, wherein the peptide ligand is capable of binding a cell surface component; and the peptide ligand and fatty acid are covalently bonded to X.

The liposome complex is capable of fusing with a cellular plasma membrane at a pH of about 5.0. In one embodiment, the peptide ligand may be 5 to 40 amino acids in length. The peptide ligand may be a receptor antagonist, which may bind a G-protein coupled receptor, for example. The ligand particularly may bind a receptor for endothelin, angiotensin II, neuropeptide Y, chromogranin A, growth hormone-releasing hormone, luteinizing-hormone releasing hormone, or kinin. In some embodiments, the peptide ligand consists of the amino acid sequence set forth in any one of SEQ ID NO: 1-12.

The liposome complex may further comprise a conjugated fusion peptide, which may be a hemagglutinin fragment. The hemagglutinin fragment may comprise the amino acid sequence of SEQ ID NO: 13. In one example, the conjugated fusion protein is present at 0.05-0.5 mol % of the liposome complex.

The X moiety in the liposome complex above may comprise a cysteine residue that forms a peptide bond with the peptide ligand. In particular, two conjugated peptide ligands may covalently bind each other by a disulfide bond between the cysteine residues. For example, the X moiety may comprise a disulfide bond. In another embodiment, the X moiety may comprise sn-glycero-3-succinate. For example, the X moiety may comprise sn-glycero-3-succinate-Cys.

In some embodiments, the fatty acid constituents of the liposome complex may be 14-18 carbons in length. In a particular embodiment, the X-fatty acid component of formula (I) is 1,2-dioleoyl-sn-glycero-3-succinate-Cys. In another embodiment, the liposome constituent of the liposome complex comprises phosphatidyl serine, phosphatidyl ethanolamine, and cholesterol in a 1:1:1 molar ratio.

In another aspect, the liposome complex further comprises a therapeutic payload, which in some embodiments is a nucleic acid encoding a therapeutic protein, a drug, or a nucleic acid. The nucleic acid may down-regulate the expression of a cognate receptor for the peptide ligand, for example. In such an embodiment, the nucleic acid may be an antisense or siRNA molecule that specifically down-regulates mRNA encoding the cognate receptor, for example. Alternatively or additionally, the therapeutic payload may be a nucleic acid that encodes a therapeutic protein. The nucleic acid encoding the therapeutic protein particularly may comprise a plasmid, and the therapeutic protein may be a cholinesterase, among other things. Alternatively or additionally, the therapeutic payload may be a therapeutic drug and an antisense oligonucleotide or siRNA.

In another aspect, a method of preparing a liposome complex, such as those above, comprises mixing the conjugated peptide ligand and lipids in an organic solvent, removing the organic solvent, and mixing said conjugated peptide ligand and lipids in an aqueous solution to form liposome complexes. The organic solvent may be chloroform or chloroform:methanol. The method may further comprise sonicating the aqueous solution following mixing. The aqueous solution may comprise 8% detergent and the method may further comprise using reverse dialysis to extract the detergent. The lipid constituent may be phosphatidyl serine, phosphatidyl ethanolamine, and cholesterol in a 1:1:1 molar ratio. As an alternative example, a method of preparing a liposome complex may comprise:

(a) preparing a solution comprising: (i) PBS; (ii) a conjugated peptide ligand; and (iii) phospholipids and cholesterol;

(b) mixing the solution; and

(c) sonicating and extruding the solution through polycarbonate filters to form the liposome complex.

The phospholipids and cholesterol in this method may be phosphatidyl serine, phosphatidyl ethanolamine, and cholesterol in a 1:1:1 molar ratio, for example.

In another aspect a pharmaceutical composition may comprise: (a) a therapeutically effective amount of a liposome complex; and (b) a pharmaceutically acceptable carrier. A method of treating a receptor-associated disorder in a patient in need thereof may comprise administering to the patient a liposome complex and/or a pharmaceutical composition comprising a liposome complex in an amount effective to treat the disorder, where the receptor-associated disorder comprises an over-abundance and/or up regulation of receptors. The receptor-associated disorder may be an endothelin receptor-associated disease, for instance, and the disease may affect lungs and/or prostate.

Both the foregoing general description and the following detailed description are exemplary and explanatory only, and are not restrictive of the invention as claimed. The accompanying drawings, which are incorporated herein by reference, and which constitute a part of this specification, illustrate certain embodiments of the invention and, together with the detailed description, serve to explain the principles of the present invention.

### BRIEF DESCRIPTION OF THE DRAWINGS

The accompanying drawings are incorporated in and constitute a part of this specification and illustrate various non-limiting embodiments. The patent or application file contains at least one drawing executed in color. Copies of this patent or patent application publication with color drawing(s) will be provided by the Office upon request and payment of the necessary fee.

FIG. 1A, FIG. 1B, and FIG. 1C depict the trafficking of a liposomal DNA payload into the nucleus of A549 lung epithelial cells.

FIG. 2 depicts binding of liposome complexes comprising a conjugated endothelin peptide ligand to two cell lines. The absolute fluorescence obtained in each case is a specific characteristic of each cell line.

FIG. 3A and FIG. 3B depict the rate of recovery of endothelin receptors following pre-exposure to unlabeled liposomes and reduction of 50% of the receptors, as a function of time for RBL mast cells and A549 lung cancer epithelial cells, respectively.

FIG. 4 depicts the correlation between the concentration of phosphatidyl serine (DOPS) in the liposome formulation and the ability of the DOPS liposomes to fuse at pH 5.0.

FIG. 5 depicts expression of a nucleic acid encoding green fluorescence protein (GFP) in a rat trachea. Test rats were given a single dose of 50 μg of GFP DNA encapsulated in a liposome complex comprising a conjugated endothelin peptide ligand by intratracheal instillation. The insert is an enlargement of the area of the tissue section outlined by the dashed line.

### DETAILED DESCRIPTION

A liposome complex is provided to target drugs and other therapeutic compounds to a particular cell. The liposome complex comprises, i.e., encapsulates, a peptide ligand that is conjugated to a lipid-soluble moiety, allowing the incorporation of the conjugated peptide ligand into the liposome complex. The fatty acid component of the conjugated peptide ligand anchors the conjugated peptide ligand in the liposome complex and exposes the peptide ligand to the surface of the liposome complex, where the peptide ligand may interact with its cognate receptor. The liposome complex comprises a conjugated peptide ligand having the following general structure:

peptide ligand-X-fatty acid (I).

### 1. Definitions and Abbreviations

“About” for the purpose of this disclosure means the range of experimental error typically associated with measuring the parameter modified by the word “about,” or ±10 percent of the parameter's disclosed value, whichever is higher.

“Cognate receptor” refers to a cell surface molecule to which the peptide ligand specifically binds. “Cognate receptor” is synonymous for all purposes with “conjugate receptor.”

“Double arm active site” refers to two conjugated peptide ligands and/or fusion peptides connected to each other through a disulfide bond contributed by a cysteine residue between the peptide ligand and a linker moiety, X in formula (I).

A “fusogenic liposome” means a liposome complex that is capable of fusing with the plasma membrane of a targeted cell. A “fusogen” is a component of a liposome complex that promotes its fusion with the plasma membrane, particular the plasma membrane of an endosome. When the fusogen is a peptide, the fusogen may be termed a “fusion peptide.” A “conjugated fusion peptide” comprises a fusogenic peptide conjugated to fatty acid by a linker moiety, X, where X is defined in the manner as in the peptide ligand conjugate of formula (I). Liposome complexes composed of equimolar amounts of PS, PE, and Chol are “fusogenic liposomes” and facilitate endosomal escape without the addition of a conjugated fusion peptide.

“Liposome complex” is used synonymously herein with a “targeted liposome.” A “liposome complex” as used herein comprises a conjugated peptide ligand having the general structure shown in formula (I). The liposome complex also contains a lipid component that forms a bilayer(s) around an internal aqueous solution. A liposome complex “incorporates” the lipid moiety of the conjugated peptide ligand, when the conjugated peptide ligand integrates into the lipid component of the liposome complex. The liposome complex also “encapsulates” a therapeutic payload that partitions into the lipid component and/or is wholly or partially soluble in the interior aqueous solution of the liposome complex.

“Specific binding” is defined functionally herein. Binding is typically measured by the association of an appropriately labeled peptide ligand to the cognate membrane receptor. Specific binding refers to binding of the labeled peptide ligand that may be displaced by an excess of unlabelled ligand. “High affinity binding” refers specifically to binding of the peptide ligand to the cognate receptor with an apparent dissociation constant of no more than about 10⁻⁴ M.

“Target cell” refers to a eukaryotic cell, e.g., an animal, mammalian, or particularly human cell, that expresses a cognate receptor on its surface. A target cell may be isolated or may be present in the body of an animal or human, to whom the liposome complex is administered.

A “therapeutic payload” means a drug and/or other therapeutically useful compound, e.g., a nucleic acid, that is encapsulated in the liposome and delivered to the interior of a target cell when the liposome fuses with the target cell.

The following abbreviations are used:

A549 Human lung epithelial cell line

Bo ACHE bovine acetylcholinesterase

Chol cholesterol

DOPC 1,2-dioleyl-sn-glycero-3-phosphocholine

DOPE 1,2-dioleyl-sn-glycero-3-phosphoethanolamine

DOPS 1,2-dioleoyl-sn-glycero-3-[phosphor-1-serine]

ET_{A}; ET_{B} endothelin receptor types A and B, respectively

GFP green fluorescence protein

GPCR G-protein coupled receptor

Hu AChE human acetylcholinesterase

Hu BuChE human butyrylcholinesterase

LC-SPDP succinimidyl 6-(3-[2-pyridyldithio]-propionamido)hexanoate

LHRH luteinizing-hormone releasing hormone

MPS mononuclear phagocytic system

OP organophosphate

PBS phosphate buffered saline

PE phosphatidyl ethanolamine

PS phosphatidyl serine

RBL rat peritoneal mast cells

RES reticuloendothelial system

siRNA small-interfering ribonucleic acids

SPDP N-succinimidyl 3-(2-pyridyldithio)-propionate

Tₘ gel-to-liquid crystalline phase transition temperature of a lipid bilayer

### 2. Liposome Complexes

An exemplary liposome complex is fusogenic, and the lipid composition of the liposome accordingly is selected to promote fusion of the liposome complex with the plasma membrane. To this end, the molar percent of phosphatidyl serine in the liposome may be about 10-40% of total lipids on a molar basis, or 30-33 mole percent of total lipids. In one embodiment, the molar percent of phosphatidyl serine is about 33%. In another embodiment, the liposome may comprise phosphatidyl serine (PS), phosphatidyl ethanolamine (PE), and cholesterol (Chol) in a molar ratio of about 1:1:1, which form a “fusogenic liposome.” In another embodiment, the melting temperature (Tₘ) of the lipids constituting the liposome may be close to the melting temperature of the plasma membrane. In another embodiment, the lipids have a Tₘ of about 41-42° C. The relationship between lipid composition of liposomes and melting temperature is well known in the art. Melting temperature of the liposomes also may be determined experimentally using scanning calorimetry or any other similarly well established method known to the skilled artisan. Phospholipids and other lipids suitable for use include natural phospholipids and/or such lipids that can be purchased from well known commercial sources, such as Avanti Polar Lipids (Alabaster, Ala.).

Liposome formation technology is well established in the art, and the details thereof will not be repeated herein. In general, the liposomes disclosed herein can be prepared by combining the components of the liposome, especially phospholipids, in an aqueous solution, then manipulating the solution to form the liposome. The liposomes thus formed may be multilamellar vesicles or unilamellar vesicles of various sizes, according to well established relationships between the method of preparation and the final physical characteristics of the liposome. Exemplary liposome formation techniques include: mixing the liposome components in organic solvent (e.g., chloroform or chloroform:methanol), slowly evaporating the organic solvent to form a film of the phospholipid, adding an aqueous solution, and thoroughly mixing the aqueous solution to form multilamellar liposomes. A further step may include sonicating the multilamellar vesicles to create small unilamellar vesicles. For example, in those cases where the conjugated peptide ligands are heavily charged and insoluble in a methanol:chloroform mixture, liposome complexes can be formulated by mixing the phospholipids and encapsulating the conjugated peptide ligands in the presence of 8% w/w detergent and using reverse dialysis method to slowly extract the detergent. The method enables the use of two or more conjugated peptide ligands simultaneously.

The conjugated peptide ligand may be inserted into the liposome complex as the liposome is being formed. For example, a liposome complex may be prepared by a method comprising (a) preparing a solution comprising: (i) PBS; (ii) a conjugated peptide ligand; and (iii) phospholipids (e.g., DOPE, DOPS) and cholesterol, (b) mixing the solution, and (c) sonicating and extruding the solution through polycarbonate filters to form the liposome complex at the required size.

Cells of the reticuloendothelial system (RES) and the mononuclear phagocytic system (MPS), particularly Kuppfer cells, are known to remove liposomes within minutes to hours of intravenous injection. In one embodiment, the lipid composition and size of the present liposome complexes are chosen to avoid the RES and/or to lengthen the circulation time of the liposome complexes. RES-avoiding, long-circulating liposomes are known in the art and described, for example, in Oku et al., “Long-circulating liposomes,” Crit. Rev. Ther. Drug Carrier Syst. 11(4): 231-70 (1994). In another embodiment, such a liposome complex has a diameter of about 100-200 nm.

### 3. Peptide Ligands

The peptide ligand component of the liposome complex brings the liposome complex in proximity to the target cell, facilitating the fusion of the liposome complex with the plasma membrane of the cellular target. The liposome complex itself packages drugs or other therapeutics that are delivered to the interior of the cell upon fusion of the liposome complex with the plasma membrane.

Peptide ligands generally specifically bind a cell surface molecule on the target cell. In one embodiment, the cell surface molecule is uniquely expressed by the target cell type. In another embodiment, the cell surface molecule is a receptor, such as a transmembrane receptor. The cell surface molecule to which the peptide ligand specifically binds is herein termed the “cognate receptor” of the peptide ligand. Cognate receptors include membrane associated proteins, carbohydrates, glycolipids, or the like, that specifically bind a peptide ligand.

The peptide ligand in one embodiment is a competitive inhibitor of its cognate receptor. In this embodiment, the peptide ligand of the liposome complex binds, but does not activate, the cognate receptor on the target cell. Alternatively or additionally, the peptide ligand may down-regulate the cognate receptor. Receptors may be down-regulated, because the peptide ligand blocks the binding of the receptor's normal ligand and/or because the binding of the peptide ligand promotes rapid internalization of the cognate receptor. Peptide ligands useful for these purposes include hormones, growth factors, or cognate receptor-binding fragments thereof that are 5 to 40 amino acids in length.

The cognate receptor may be a G-protein coupled receptor (GPCR), for example. Suitable GPCRs include, but are not limited to, receptors for bombesin; bradykinin; endothelin; hepatocyte growth factor; melanocortins; neuropeptide Y; opsins; somatostatin; tachykinins; vasoactive intestinal polypeptide family and vasopressin; chemokines; and peptide hormones (e.g., calcitonin, C5a anaphylatoxin, follicle-stimulating hormone, gonadotropic-releasing hormone, neurokinin, thyrotropin-releasing hormone, and oxytocin).

One such GPCR is the endothelin receptor system. The endothelin receptor system consists of two GPCRs, three endothelin peptide ligands, and two activating peptidases. The system is known for its pharmacological complexity, which is reflected by the expression of its components that have a variety of physiological and pathophysiological roles. Besides a role of the endothelin receptor system in maintaining homeostasis in the body, the pathophysiology of these receptors is associated with diseases such as vascular hypertension, atherosclerosis, and vasospasm. See Schiffrin et al., “Vascular endothelin in hypertension,” Vascul. Pharma. 43: 19-29 (2005). In the kidney, endothelin controls water and sodium excretion as well as acid-base balance, and participates in acute and chronic renal failure. See Orth et al., “Endothelin in renal diseases and cardiovascular remodeling in renal failure,” Inter. Med. 40: 285-91 (2001). In the heart, endothelin affects ionotropy and chronotropy and mediates cardiac hypertrophy and remodeling after congestive heart failure and renal failure. See Orth (2001); Ram, “Possible therapeutic role of endothelin antagonist in cardiovascular disease,” Am. J. Ther. 10: 396-400 (2003). In lung, endothelin regulates the tone of airways and blood vessels and is involved in the development of pulmonary hypertension. The endothelin system also is widely expressed in brain cells, and the altered expression of endothelin in reactive astrocytes has been observed in many pathological conditions of the brain. See Schinelli, “Pharmacology and physiopathology of the brain endothelin system: an overview,” Curr. Med. Chem. 13: 627-38 (2006). Additionally, functional endothelin receptors are expressed in tumors of the brain and other tissues, e.g., prostate. See Nelson, “Endothelin inhibition: Novel therapy for prostate cancer,” J. Urol. 170: S65-S57 and S67-S68 (2003); Khan et al., “Endothelin-A receptor antagonists and advanced prostate cancer,” Rev. Urol. 6: 47-48 (2004). It has been suggested the endothelin antagonists may have a therapeutic role in the pharmacological treatment of the diseases above. See id.

The cognate receptor can be expressed on the surface of the targeted cell type. In one embodiment, the cognate receptor is expressed exclusively on the targeted cell type(s). In other embodiments, the cognate receptor is expressed by a limited number of cell types, including the targeted cell type(s). The tissue expression pattern of various receptors is well known in the art. For example, when targeting cells to mast and epithelial cells of the lung, an exemplary peptide ligand may be an endothelin receptor antagonist, such as Cys-His-Leu-Asp-Ile-Ile-Trp (SEQ ID NO: 1). When targeting to kidney cells is desired, an exemplary peptide ligand may be an angiotensin II receptor antagonist, such as Glu-Gly-Val-Tyr-Val-His-Pro-Val (SEQ ID NO: 2). Additional, representative peptide ligands include, but are not limited to, the angiotensin II receptor antagonists Gly-Arg-Val-Tyr-Ile-His-Pro-Thr (SEQ ID NO: 3) and Gly-Arg-Val-Tyr-Val-His-Pro-Ala (SEQ ID NO: 4). When targeting heart cells, an exemplary peptide ligand may be a neuropeptide Y receptor antagonist Ala-Arg-Tyr-Tyr-Ser-Ala-Leu-Arg-His-Tyr-Ile-Asn-Leu-Ile-Thr-Arg-Gln-Arg-Tyr (SEQ ID NO: 5). When targeting pancreatic cells, an exemplary peptide ligand may be a chromogranin A fragment, such as residues 286-301 of chromogranin A: Glu-Glu-Glu-Glu-Glu-Met-Ala-Val-Val-Pro-Gln-Gly-Leu-Phe-Arg-Gly-NH₂ (SEQ ID NO: 6). When targeting pituitary cells, an exemplary peptide ligand may be a fragment of growth hormone-releasing hormone, such as Tyr-Ala-Asp-Ala-Ile-Phe-Thr-Ser-Asn-Tyr-Arg-Lys-Val-Leu-Gly-Gln-Leu-Ser-Ala-Arg-Lys-Leu-Leu-Gln-Asp-Ile-Met-Ser-Arg-NH₂ (SEQ ID NO: 7). When ovary cells are targeted, an exemplary peptide ligand may be a fragment of luteinizing-hormone releasing hormone (LHRH), such as pyroGlu-His-Trp-Ser-Tyr-Gly-Leu-Arg-Pro-Gly-CONH₂ (SEQ ID NO: 8). When lung cells are targeted, an exemplary peptide ligand may be kinin, comprising Cys-Asn-Ala-Glu-Val-Tyr-Val-Val-Pro-Trp-Glu-Lys-Lys (SEQ ID NO: 9), such as Leu-Asp-Cys-Asn-Ala-Glu-Val-Tyr-Val-Val-Pro-Trp-Glu-Lys-Lys-Ile-Tyr-Pro-Thr-Val-Asn-Cys-Gln-Pro-Leu-Gly-Met (SEQ ID NO: 10), the Cys residues of which can form an intramolecular disulfide bond. The peptide ligands listed above are non-limiting examples of peptide ligands. In each case, the length of the peptide ligand may be adjusted by including more or less amino acids than found in the corresponding naturally occurring sequence. For example, the endothelin receptor ligand of SEQ ID NO: 1 may comprise fewer amino acid residues than are present in SEQ ID NO: 1. The peptide ligand may consist of the amino acid sequence His-Leu-Asp-Ile-Ile-Trp (SEQ ID NO: 11) or Leu-Asp-Ile-Ile-Trp (SEQ ID NO: 12), for example.

Rapid internalization of the peptide ligand in a binding complex with the cognate receptor, e.g., a GPCR, promotes rapid co-internalization of the present liposome complexes. Because the present liposome complexes are rapidly removed from circulation by co-internalization with the cognate receptor, the present liposome complexes advantageously will avoid internalization by non-target cells, such as Kuppfer cells and other cells of the RES or MPS.

Receptor-mediated internalization (i.e., endocytosis) of the liposome complexes typically results in accumulation of the liposome complexes in cellular endosomes. Endosomes are intracellular vesicles generally formed by invagination and pinching off of the plasma membrane. The endosome interior rapidly acidifies to a pH of about 5.0, typically followed by enzymatic degradation of the endosomal contents in lysosomes. To ensure release of the contents of internalized liposome complexes to the interior of the target cell, i.e., endosome escape, the liposome complex can be designed to fuse with the endosome membrane under acidified conditions.

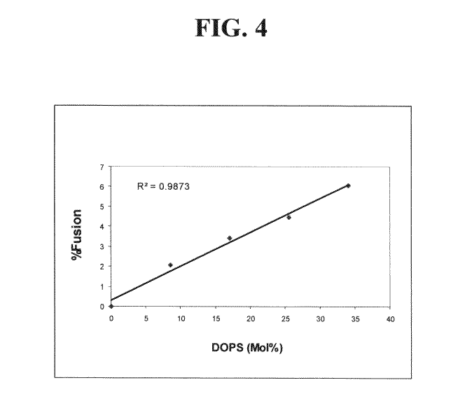

Endosome escape may be facilitated by incorporating a “fusogen” into the liposome complex or by increasing PS concentration to about 10-40%, e.g., 30-33%. In one embodiment, the fusogen is a fusion peptide (e.g., the 23 amino acid peptide fragment of hemagglutinin of SEQ ID NO: 13). The fusion peptide may be conjugated to fatty acids via covalent bonding to a linker moiety in the same manner as the peptide ligand conjugate of formula (I). The fusion peptide may be incorporated into the liposome complexes at various concentrations. Liposome complexes comprising the conjugated hemagglutinin fusion peptide efficiently fused to naked liposomes at pH 5.0, but showed practically no fusion at pH 7.4. Adding a conjugated peptide ligand to the same liposome complex does not interfere with the fusogenic capacity of the liposomes at low pH. PS likewise promotes fusion and endosome escape. FIG. 4 shows a correlation between the concentration of phosphatidyl serine (DOPS, in this instance) in the liposome formulation and the ability of liposomes to fuse at pH 5.0. As depicted in FIG. 4, PS facilitates liposome fusion over a broad range of mole percent of total lipids.

The liposome complex may comprise just the conjugated peptide ligand itself. In this embodiment, diseases and/or conditions treatable by the present compositions include receptor-associated diseases or disorders in which a patient can benefit from the internalization and/or down-regulation of cognate receptor. That is, the disease or disorder may be related to the expression of a receptor, such that internalization and/or down-regulation of the receptor treats the receptor-associated disease or disorder. Receptor-associated diseases or disorders include those in which the cognate receptor is over-abundant or up-regulated, such that the number of receptors on the cell surface or the activity of the receptors bound by ligand is abnormally high. In one non-limiting example, the receptor-associated disease is prostate cancer, which is associated with an abnormal expression of the endothelin receptor.

The liposome complex may further comprise a conjugated fusion peptide. Liposome complexes containing both conjugated fusion peptides and conjugated peptide ligands at concentrations of 0.05-0.5 mol % are found to be very effective. Liposome complexes composed of equimolar amounts of PS, PE, and Chol are fusogenic and facilitate endosomal escape without the addition of a conjugated fusion peptide.

The liposome complex further may encapsulate a therapeutic payload (e.g., drugs or therapeutic nucleic acids) to be delivered to a target cell. Accordingly, also provided are compositions and uses thereof to treat diseases and/or conditions by delivering drugs or other suitable pharmaceutical formulations to target cells. In one non-limiting example, the therapeutic payload is a nucleic acid that down-regulates the expression of the cognate receptor. The nucleic acid in this embodiment may be an antisense nucleic acid, siRNA, or the like, that specifically recognizes and down-regulates expression of mRNA encoding the cognate receptor.

### 4. Conjugated Peptide Ligands

In one aspect, peptide ligands in the present disclosure are conjugated to fatty acids to form a “conjugated peptide ligand” having a structure according to formula (I):

peptide ligand-X-fatty acid (I),

where X is a linker moiety. The peptide ligand is covalently bound to the linker moiety (signified in formula (I) by “peptide ligand-X”) by the ligand's N-terminal amine group, C-terminal carboxyl group, or reactive group provided by the side chain of an amino acid residue within the peptide sequence, e.g., a sulfhydryl group provided by a cysteine residue. The nature of the covalent bond between the peptide ligand and the linker moiety is determined by the chemical reactive group(s) of the linker moiety. One or two fatty acids may be covalently bonded to the linker moiety. The fatty acid increases the hydrophobicity of the conjugated peptide ligand, allowing the conjugated peptide ligand to partition into the lipid component of the liposome complex. The fatty acids anchor the conjugated peptide ligand, orienting the peptide ligand to the surface of the liposome complex, where the peptide ligand is then free to interact with its cognate receptor on the target cell.

The fatty acid may be any fatty acid with sufficient length to anchor the conjugated peptide ligand in the liposome complex. In one embodiment, the fatty acids are 14 to 18 carbons in length. Examples of suitable fatty acids include myristic acid, myristoleic acid, palmitic acid, palmitoleic acid, stearic acid, oleic acid, linoleic acid, and α-linolenic acid. The conjugated peptide ligand may contain two fatty acids, which may be the same or different.

In one aspect, the N-terminal of the peptide ligand forms an amide bond with the carboxyl group of 1,2-di-(fatty acid)-sn-glycero-3-succinate (i.e., X is glycero-3-succinate). When both fatty acids are dioleoyl, for example, the molecule thus formed is soluble in methanol or methanol-chloroform. Liposome complexes comprising this conjugate thus can be formed from components dissolved in a methanol or methanol:chloroform solvent. Liposome complexes comprising the endothelin fragment of SEQ ID NO: 1, for example, conjugated to 1,2-dioleoyl-sn-glycero-3-succinate (Avanti Polar Lipids, Alabaster, Ala.) serve as a competitive antagonist to endothelin receptors. The targeted liposomes bound endothelin receptors on lung epithelial and mast cells, followed by rapid desensitization and internalization of the liposome complex bound to the endothelin receptor. The same liposome complexes, when loaded with cDNA, released their therapeutic payload into the cytoplasm after internalization. The cDNA is directed to the nucleus of the cell. Targeting also can be accomplished when the liposome complexes further comprise a fusogen, such as a conjugated fusion peptide.

In another embodiment, a cysteine residue is added between the peptide ligand and the linker moiety, thereby providing additional spacing between the peptide ligand and the liposome surface. In this embodiment, the N-terminal amino group of the cysteine residue may be conjugated to the linker moiety in the same manner as the N-terminal residue of the peptide ligand. The cysteine residue further enables the formation of disulfide bonds between two conjugated peptide ligands and/or fusion peptides. Two disulfide-bonded moieties are termed a “double arm active site.” The efficiency of targeting and/or fusion may be improved using double arm active sites. Further, the use of such double arm active sites facilitates the preparation of the targeting liposomes at a controlled concentration. One non-limiting example of a compound of formula (I) that comprises a Cys residue is a compound comprising 1,2-dioleoyl-sn-glycero-3-succinate-Cys, where the amino group of Cys forms an amide bond with the carboxyl group of 1,2-dioleoyl-sn-glycero-3-succinate.

In another aspect, peptide ligands may be conjugated to N-succinimidyl 3-(2-pyridyldithio)-propionate (SPDP) or a long chain derivative, succinimidyl 6-(3-[2-pyridyldithio]-propionamido)hexanoate (LC-SPDP) (Thermo Fisher Scientific Inc., Rockford, Ill.), depending on the desired spacing between the peptide ligand and the liposome surface. The structure of SPDP is shown as formula (II) below:

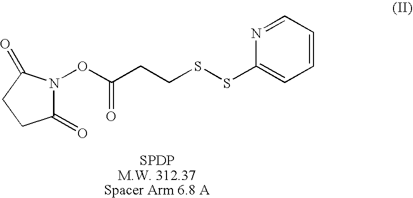

The N-hydroxysuccinimide ester moiety reacts with amine groups to form a stable amide bond at one end of the spacer arm. The other end of the spacer arm terminates with a pyridyl disulfide group that will react with sulfhydryls to form a reversible disulfide bond. The amine group may be contributed a phospholipid, e.g., phosphatidyl ethanolamine (PE). In this embodiment, the constituent fatty acids of PE are the “fatty acid” moiety of the conjugated peptide ligand of formula (I), and the sn-glycero-3-phosphoethanolamine head group of PE is considered part of the linker group, X. In this embodiment, the PE-pyridyl disulfide derivative can be reacted with a sulfhydryl to form a disulfide bond and release pyridine-2-thione, which can be measured at 343 nm. See LIPOSOMES: A PRACTICAL APPROACH, R.R.C. New, ed., IRL Press, Oxford, UK (1990).

The sulfhydryl can be contributed by a Cys residue at a terminus or within the amino acid sequence of the peptide ligand. Alternatively, the sulfhydryl can be formed by first reacting an amine group of the peptide ligand with SPDP to form a pyridyl disulfide derivative of the peptide ligand. The pyridyl disulfide derivative can then be reacted with a reducing agent, such as dithiothreitol, releasing pyridine-2-thione and leaving a reactive sulfhydryl group. The conjugated peptide ligand thus formed will be covalently bound to the linker via a disulfide bond. Because disulfide bonds generally are less stable than amide bonds in vivo, the conjugated peptide ligand comprising 1,2-di-(fatty acid)-sn-glycero-3-succinate described above is expected to provide a more stable conjugated peptide ligand, when used in a therapeutic composition or method.

### 5. Payload-Containing Targeting Liposomes

Provided herein are compositions and methods related to the targeted delivery of active agents, such as drugs, genes or short antisense nucleotides into a diseased cell or tissue. Also provided herein are compositions and methods for a liposome-based agonist or antagonist that enables targeted delivery of drugs, genes or short antisense nucleotides into a diseased tissue, along with down-regulation of the number of target receptors. In one aspect, the combined action of both cognate receptor antagonism (e.g., a reduction in the number of receptors) by the peptide ligand and liposome payload (e.g., a reduction or blockade of cognate receptor synthesis) can be used to accelerate patient homeostasis and recovery.

It is further shown herein that the conjugated peptide ligand attaches to both the inner and outer surface of the liposomes, and that the presence of the conjugated peptide ligand on the surface of the liposomes improves the targeting efficacy of the liposome complex. In one aspect, the presence of the conjugated peptide ligand on the surface of the liposomes improves the endothelin-receptor targeting capabilities of the targeted liposome, when compared to the endothelin receptor targeting capabilities of the liposome without the conjugated peptide ligand.

The present invention discloses compositions and methods for formulating targeted liposomes for gene, oligonucleotide, or drug delivery. In those cases where the peptides are heavily charged and the conjugated peptide ligand is insoluble in methanol:chloroform, liposome complexes can be formed by mixing the phospholipids and the conjugated peptide ligands in the presence of 8% detergent and using reverse dialysis method to slowly extract the detergent to form liposomes.

In one aspect, the liposome complex encapsulates a therapeutic payload. In an exemplary embodiment, the therapeutic payload comprises at least one of: (a) a gene encoding a therapeutic protein; (b) an antisense oligonucleotide; and (c) a therapeutic drug.

Gene therapy is emerging as a clinically viable therapeutic regimen for genetic, neoplastic and infectious diseases. Originally, gene therapy was aimed at partial or total replacement of a diseased gene, and, thus, at reducing the pathological manifestations of this gene. In recent years a number of models for gene therapy against various pathological manifestations have been presented. These include gene replacement, gene therapy and oligonucleotide or siRNA therapy. Several DNA transfection vectors have been used, including virus vectors and bacterial plasmid DNA.

The plasmid DNA transfection approach, using a liposome complex as the gene delivery system, has several advantages, especially when only transient (e.g., days or weeks) treatment is required (e.g., destruction of tumor cells or induced protection against inhaled chemical warfare agents). Among the advantages of the present liposome complexes are the convenience of use and the ability to administer the therapeutic payload in the form of an inhaled aerosol. The present composition advantageously can be safely administered repeatedly, because no viral structures are needed in the composition and because the natural phospholipid components are non-immunogenic. Furthermore, liposome-encapsulated genes can be lyophilized to achieve a prolonged shelf-life, or liposomes can be prepared as two separate lyophilized components that are mixed before use, as described herein. When the therapeutic payload is a nucleic acid, such as a DNA vector or plasmid, the nucleic acid can be pre-complexed with protamine to facilitate encapsulation and delivery of the nucleic acid to the nucleus of the target cell.

Bacterial plasmid DNA used in the present liposome complexes does not integrate with the host genome and thus is not limited to use and utility with a specific cell. This transient effect provides better control over the degree and duration of the expression of the delivered coding sequence.

In an exemplary embodiment, the therapeutic payload in a liposome is a gene encoding a therapeutic protein (e.g., a cholinesterase). In this embodiment, the gene encoding the therapeutic protein may be part of a plasmid. In another embodiment, the therapeutic payload is an antisense oligonucleotide or siRNA. In still another embodiment, the therapeutic payload is a therapeutic drug.

In one embodiment, a liposome comprises a conjugated endothelin peptide ligand and a therapeutic payload comprising a gene encoding a cholinesterase. In this embodiment, the cholinesterase gene may encode human butyrylcholinesterase (Hu BuChE) or bovine acetylcholinesterase (Bo AChE). When the cholinesterase gene is taken up and expressed by target cells, the cholinesterase will be secreted into the lining fluid of the lungs, where it can scavenge inhaled organophosphate (OP) compounds, for example, and reduce the amount of OP that enters the blood. In the transfected lungs, the cholinesterase activity will be restricted mainly to the lining fluid of the lungs, and, thus, minimize the adverse effects while providing improved protection. This method, and related methods, are described in detail in pending patent application U.S. Ser. No. 11/339,404, which is incorporated herein by reference in its entirety.

For example, injection of exogenous Hu BuChE provided protection against multiple lethal doses of nerve agents in mice (Ashani et al., “Butyrylcholinesterase and acetylcholine-esterase prophylaxis against soman poisoning in mice,” Biochem. Pharmacol. 41: 37-41 (1991)), monkeys (Raveh et al., “The stoichiometry of protection against soman and VX toxicity in monkeys pretreated with human butyrylcholinesterase,” Toxicol. Appl. Pharmacol. 145: 43-53 (1997)), as well as protection against multiple inhaled lethal doses in guinea pigs (Allon et al., “Prophylaxis against soman inhalation toxicity in guinea pigs by pretreatment alone with human serum butyrylcholinesterase,” Toxicol. Sci. 43: 121-28 (1998)). Allon (1998) showed that only 25% of the inhaled OP actually reached the blood, while the remaining 75% of inhaled OP was either exhaled (25%) or retained in the lung.

The cDNA sequences have been cloned for both Hu ACHE and Hu BuChE. See, e.g., U.S. Pat. No. 5,595,903 and U.S. Pat. No. 5,215,909, which are hereby incorporated by reference herein, to the extent that teach Hu AChE and Hu BuChE nucleotide and protein sequences, vector and host cells comprising the encoding nucleic acids, and methods of making and using the nucleic acids are. Such cDNA sequences can be incorporated into suitable vectors, such as bacterial plasmids, for expression in vitro or in vivo. The vectors can be encapsulated within the liposomes disclosed herein, and administered to a patient in need thereof to deliver the genes to lung cells, where the proteins can be expressed to provide a therapeutic treatment to a patient suffering from the effects of nerve agents.

### 6. Use and Administration of Liposome-Containing Compositions

For therapeutic uses, the liposome complexes set forth herein can be administered to a patient in need of treatment according to protocols already well established in the art. In general, a therapeutically effective amount of the liposome complexes is combined with a pharmaceutically acceptable carrier. A “therapeutically effective amount,” as used herein, varies depending upon the disorder, each specific patient and other well known factors such as age, weight, etc., and thus must be determined empirically in each case. This empirical determination can be made by routine experimentation. Typically, though, the liposome components may be used at a ratio of about 200:1 w/w, e.g., 100-300:1 w/w, compared to the therapeutic payload. A typical therapeutic dose of the liposome composition is about 5-100 mg per dose, e.g., 10 mg per dose.

In general, the liposome composition can be administered to the patient by any customary administration route, for example, orally, by injection or by inhalation. In one aspect, the liposome complex is administered to a patient intravascularly. A liposome useful for intravascular administration can be a small unilamellar liposome, or may be a liposome comprising PEG-2000. In another aspect, the liposome is administered by inhalation to the respiratory tract of a patient to target the trachea and/or the lung of a patient. In this embodiment, a commercially available nebulizer may be used to deliver a therapeutic dose of the liposome complex in the form of an aerosol.

In the case where the disorder is OP exposure, and the therapeutic payload comprises a gene encoding a cholinesterase, the liposome complex is administered from about 50 μg to about 500 μg of DNA per dose, but this range can be adjusted as necessary. In a rat model, the administration of 50-100 μg of DNA had no adverse or toxic effects. The pharmaceutically acceptable carrier is any carrier known or established in the art. Exemplary pharmaceutically acceptable carriers include, but are not limited to, sterile pyrogen-free water and sterile pyrogen-free saline solution.

In a similar manner, the compositions and methods disclosed herein can be used for gene therapy for cystic fibrosis and other genetic disorders of the lung. In one embodiment, the liposome complex comprises a therapeutic payload that comprises a gene encoding a therapeutic protein effective against cystic fibrosis or other genetic disorder of the lung. In another embodiment, the therapeutic payload comprises a therapeutic drug effective against cystic fibrosis or other genetic disorder of the lung.

It will be understood, based on the disclosure set forth herein, that by selecting the proper targeting peptide ligand, the methods and compositions can also be applied for targeting drugs or genes in general, to any affected tissue. For example, the targeting peptide ligand and the method for conjugating the peptide ligand can be used to prepare a liposome complex that is specific to a cell and/or tissue other than the lung (e.g., liver, intestine, heart). It will also be understood that the method of administration of a targeted liposome can vary based on the target (e.g., intravenous, oral, peritoneal). It is expected that the method of administration will affect the dose required for a therapeutic effect due to the differences physiological condition in each route and tissue.

In an embodiment, an endothelin-targeted liposome is disclosed herein for treatment of an endothelin receptor-related disease or disorder. Such disorders include, but are not limited to, vascular hypertension, atherosclerosis and vasospasm, acute and chronic renal failure, cardiac hypertrophy and cardiovascular remodeling after congestive heart failure or renal failure, pulmonary hypertension, infarcts, lacuane, traumatic conditions, Alzheimer's disease and inflammatory disease of the brain, tumors of the brain and of other organs (e.g., prostate). It will be understood that these, as well as any other disease or disorder associated with over-expression or hyper-activity of endothelin receptors can be treated with the methods and compositions disclosed herein.

### EXAMPLES

### Example 1

### Preparation of Liposome Complexes

### Materials and Methods

Cholesterol; 1,2-dioleyl-sn-glycero-3-phosphocholine (DOPC); 1,2-dioleyl-sn-glycero-3-phosphoethanolamine (DOPE); and 1,2-dioleoyl-sn-glycero-3-[phosphor-1-serine] (DOPS) were purchased from Avanti Polar Lipids (Alabaster, Ala.). Fluorescence markers, Texas Red, Rhodamine B (R18), and TOTO-1, were purchased from Molecular probes (Eugene, Oreg.). Specific peptides were synthesized by SynPep (Dublin, Calif.) or Global Peptide (Fort Collins, Colo.). Protamine sulfate was purchased from Sigma Chemical Co. (St. Louis, Mo.).

### Liposome Formulations

Liposomes were prepared as follows: cholesterol/DOPC/DOPE or cholesterol/DOPE/DOPS at a molar ration of 1:1:1 were prepared in 10 mM phosphate buffer saline (PBS), pH 7.4. The conjugated endothelin peptide ligand (1,2-dioleoyl-sn-glycero-3-succinate-Cys-His-Leu-Asp-Ile-Ile-Trp-COOH, which amino acid sequence is set forth in SEQ ID NO: 1) was added to the mixture at 0.2-0.5 mol %. Fluorescence markers for the lipid layer (0.05-0.1% Texas Red) and for fusion test (R18) were incorporated into the lipid layer during the formation of the initial lipid mixture. Fusion liposomes were tested using cholesterol/DOPC/DOPE liposomes labeled with R18 (6 mol %; ˜90% quenching).

### Liposome Preparation

Liposomes were composed of equimolar amounts of DOPE, DOPS (or POPC) and cholesterol dissolved in chloroform. Conjugated targeting peptide ligand at a concentration of 0.2-0.5 mol % was added to the mixture unless mentioned otherwise. The solvent was removed by rotary evaporation at 40° C. for 2 hours. The dried lipid film was hydrated in PBS solution pH 7.4 for 2 hours, using an extended arm shaker. The multilamellar vesicles were reduced in size to 200-300 nm by gentle ultrasonic treatment using a Model 75 HT (VWR Scientific) for 30 seconds. Liposomes were then subjected through two freeze and thaw cycles followed by extrusion through 200 nm polycarbonate filters using an extrusion device (Lipex Biomembranes, Vancouver, Canada). Liposomes were then lyophilized overnight (Model 4 KBTXL-75) and kept under nitrogen in sealed bottles until use. The size of the liposomes can be further reduced using additional freeze and thaw cycles, followed by extrusion through 100 nm and 50 nm polycarbonate filters. Payload (drug, DNA or protein) dissolved in minimum amount of buffer is mixed with the dry empty liposomes (30 min) before use, then brought to final concentration for treatment. Utilizing this method achieves high encapsulation efficiency and long shelf-life of the resulting liposome complexes.

### DNA Encapsulation

DNA plasmid was complexed with either synthetic peptide containing the nuclear localization signal at ratios of 30:1 to 5:1 (w/w) or protamine at a ratio of 1:3 for 30 minutes in a total volume of 150-250 μl. The DNA complex, unlabeled or labeled with 0.1% of TOTO-1, was added to lyophilized liposomes, labeled or unlabeled with Texas Red, and shaken gently until all the liposomes were wet, followed by incubation at room temperature for 30 minutes. The lipid:DNA ratio was 200:1 (w/w). Following this procedure about 95-99% of the DNA was encapsulated. The encapsulated DNA was then brought to final working concentration.

Trafficking of Liposomes and their Payload

Human lung epithelial carcinoma cell line (A549) and rat peritoneal mast cells (RBL; ATCC, Manassas, Va.) were used to follow the fate of liposomes and their payload. Cells were incubated with encapsulated plasmid for different time periods followed by wash (in PBS) and fixation with 4% formalin. The locations of fluorescently labeled phospholipids and DNA were observed under a Confocal laser scanning microscope.

### Time Course of Down-Regulation of Endothelin Receptor

Rat peritoneal mast cells (RBL) were pre-incubated with non-labeled empty liposomes comprising a conjugated peptide ligand for 30 minutes. Cells were then washed and incubated for 15 min with Texas Red-labeled liposomes comprising a conjugated peptide ligand at different time periods post pre-incubation. Cells were then washed and incubated in 0.5 ml of 1N NaOH for 60 min. The amount of fluorescence was compared to cells pretreated with PBS alone. Cells pre-incubated with liposomes comprising conjugated fusion peptide were used as controls.

### The Fate of Phospholipids and DNA Payload Following Exposure to RBL Cells

Rat peritoneal mast cells (RBL) were incubated with liposomes that were labeled with Texas red and encapsulated DNA labeled with TOTO1, comprising a conjugated peptide ligand. Cells were washed and fixed at 5, 30, and 120 min following initiation of exposure.

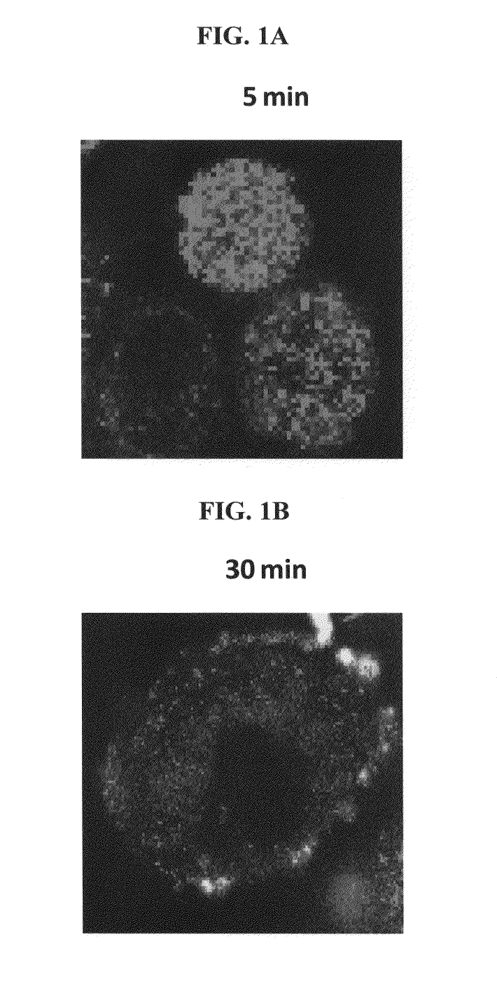

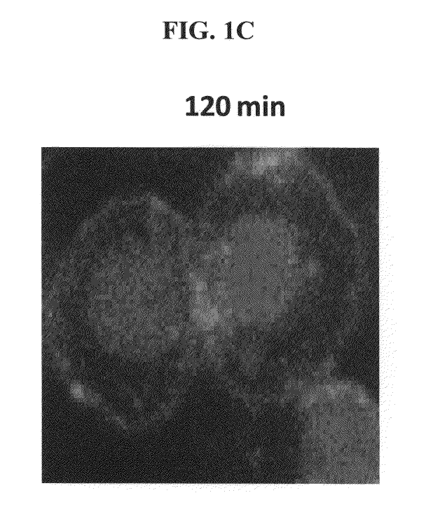

Five to ten minutes following exposure of cells to targeted liposomes, the cells were coated with liposome complexes bound to the surface of the cells as shown in FIG. 1A. Binding of the liposome complexes to the endothelin receptors was followed by fast internalization of the liposome complexes through a receptor-mediated endocytosis process. Thirty minutes post exposure, most of the liposome complexes were in the cytoplasm, and the DNA was released from the liposome complexes due to the fusion of pH-activated liposome complexes with the endosomal membrane (FIG. 1B). At 2-3 hours post-exposure, most of the DNA accumulated in the nucleus, and the phospholipids reincorporated into the cells' membrane (FIG. 1C).

### Binding Curves of Liposome-Base Endothelin Antagonist

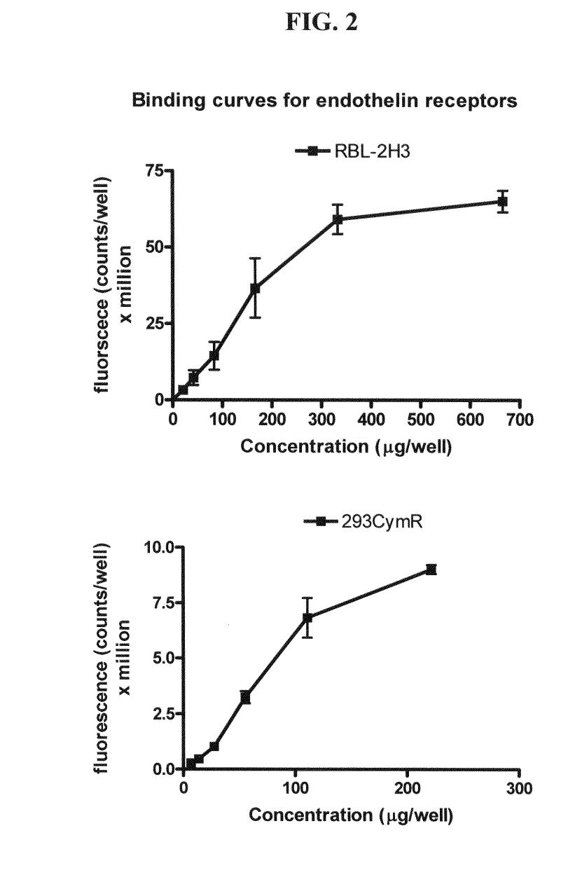

FIG. 2 shows the binding curves of liposome-based endothelin antagonist to two different cell lines; RBL-2H3 rat mast cells and prostate cancer 283CymR. A ten-fold difference was observed in the binding properties of these two cell lines, probably representing variation in the number of endothelin receptors.

### Time Course of Recovery of Endothelin Receptors

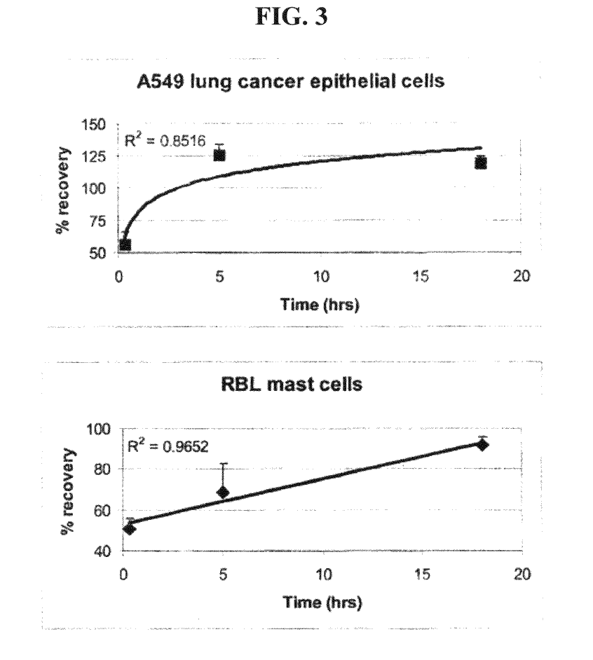

FIG. 3 illustrates the recovery, over time, of the endothelin receptors after internalization following treatment with targeted liposomes. The recovery of receptors in RBL cells was not complete even after 18 hours (FIG. 3B). On the other hand, a 100% recovery was observed in A549 human epithelial lung carcinoma cells in about 2.5 hours (FIG. 3A).

### Fusogenicity of DOPS Liposomes

FIG. 4 illustrates the correlation between the concentration of phosphatidyl serine in the liposome formulation and the ability of the liposomes to fuse at pH 5.0. The fusogenicity of liposomes containing 33 mol % phosphatidyl serine was the same as fusogenicity of liposomes containing a conjugated fusion peptide. The phosphatidyl serine in this instance was DOPS.

### In Vivo Expression of a Therapeutic Payload

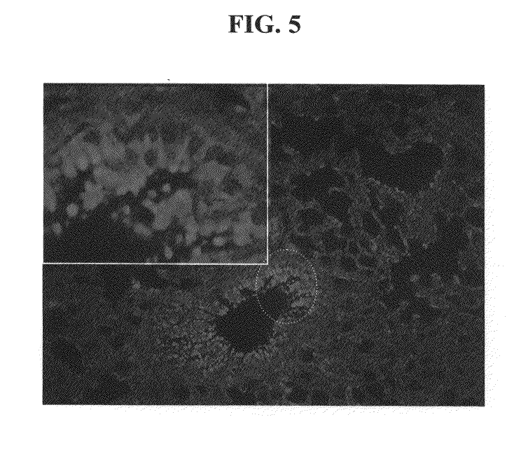

A liposome complex was administered to the trachea of a rat by intratracheal instillation. The liposome complex comprised a conjugated endothelin peptide ligand, where the endothelin peptide ligand had the amino acid sequence of SEQ ID NO: 1 and was conjugated to 1,2-dioleoyl-sn-glycero-3-succinate. The payload of the liposome complex comprised 50 μg of a nucleic acid encoding GFP. A single dose of the liposome complex was administered, and the rat was sacrificed five days later. Sections of rat tracheal tissue were obtained and fluorescence was determined using standard techniques. A fluorescence micrograph is shown in FIG. 5. GFP expression was observed around the lining of the lung, as shown. The results demonstrate that a single dose of a composition of liposome complexes comprising as little as 50 μg DNA is sufficient to obtain stable in vivo expression of a nucleic acid.

### DISCUSSION

It is shown herein that targeted liposomes containing a 7 amino acid endothelin peptide ligand conjugated to 1,2-dioleoyl-sn-glycero-3-succinate is a competitive antagonist to endothelin receptors. The targeted liposome binds to endothelin receptors, followed by rapid desensitization by way of phosphorylation through the G protein-coupled receptor kinase type 2. The desensitization is followed by internalization of receptor-liposome complex via caveolae and/or clathrin-coated pits, which results in a reduction in the number of receptors on the surface of the cells. There are two endothelin receptors (i.e., ET_{A} and ET_{B}); the relative number of each receptor and their fates following internalization differs between various cell types. It is known that ET_{B} receptors go through a lysosomal pathway and degradation. The recovery of receptors therefore depends on the synthesis of new receptors. In contrast, ET_{A} receptors are released and recycled back to the cell surface; therefore, their recovery should be faster than ET_{B}. The differences observed in the rate of recovery between the two cell-lines tested may be a reflection of the distribution of each of these receptors. Because each receptor activates different processes in the cell, their relative distribution on the cell's surface may be altered in various diseases. Such changes could affect the rate of recovery of the receptors (as shown in lung cancer cells in the present disclosure) and thus may affect the efficacy of treatment with antagonist alone. It is thus understood that a method comprising down-regulating endothelin receptors may further benefit from the therapeutic effect of the provided by various therapeutic payloads of the present liposome complexes.

The use of liposome complexes comprising conjugated peptide ligands can be adopted for the generation of new type of agonists and antagonists. Furthermore, utilizing this method, liposome complexes can carry more than one type of ligand, as well as deliver specific drug(s) or antisense nucleotides to target tissues.

It will be apparent to those skilled in the art that various modifications and variations can be made to the compositions and methods of using the same without departing from the spirit or scope of the intended use herein. These modifications and variations and their equivalents come within the scope of the claims. All patents and patent applications cited above are herein incorporated by reference in their entirety for all purposes.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

1. A liposome complex comprising:
(i) a conjugated peptide ligand of formula (I):
peptide ligand-X-fatty acid (I); and
(ii) a liposome comprising about 10-40 mole percent phosphatidyl serine compared to total lipids, and wherein the liposome is capable of fusing with a cellular plasma membrane,
wherein the peptide ligand is capable of binding a cell surface component in the cellular plasma membrane; and the peptide ligand and fatty acid are covalently bonded to X.

2. The liposome complex of claim 1, wherein the liposome is capable of fusing with a cellular plasma membrane at about pH 5.0.

3. The liposome complex of claim 1, wherein the peptide ligand is 5 to 40 amino acids in length.

4. The liposome complex of claim 1, wherein the peptide ligand is a receptor antagonist.

5. The liposome complex of claim 1, wherein the peptide ligand binds a G-protein coupled receptor.

6. The liposome complex of claim 1, wherein the peptide ligand binds a receptor for endothelin, angiotensin II, neuropeptide Y, chromogranin A, growth hormone-releasing hormone, luteinizing-hormone releasing hormone, or kinin.

7. The liposome complex of claim 6, wherein the peptide ligand consists of the amino acid sequence set forth in any one of SEQ ID NOS 1 and 3-13.

8. The liposome complex of claim 1, wherein X comprises a cysteine residue that forms a peptide bond with the peptide ligand.

9. The liposome complex of claim 8, comprising two conjugated peptide ligands covalently bound to each other by a disulfide bond between said cysteine residues.

10. The liposome complex of claim 1, wherein the conjugated peptide ligand is present at 0.05-0.5 mol % of said liposome complex.

11. The liposome complex of claim 1, further comprising a conjugated fusion peptide.

12. The liposome complex of claim 11, wherein the conjugated fusion peptide comprises a hemagglutinin fragment.

13. The liposome complex of claim 12, where the hemagglutinin fragment comprises the amino acid sequence of SEQ ID NO: 2.

14. The liposome complex of claim 11, wherein the conjugated fusion protein is present at 0.05-0.5 mol % of said liposome complex.

15. The liposome complex of claim 1, wherein the fatty acid of formula (I) is 14-18 carbons in length.

16. The liposome complex of claim 1, wherein X comprises a disulfide bond.

17. The liposome complex of claim 1, wherein X comprises sn-glycero-3-succinate.

18. The liposome complex of claim 17, wherein X comprises sn-glycero-3-succinate-Cys.

19. The liposome complex of claim 18, wherein said X-fatty acid component of formula (I) is 1,2-dioleoyl-sn-glycero-3-succinate-Cys.

20. The liposome complex of claim 1, wherein phosphatidyl serine is about 30-33 mole percent of total lipids.

21. The liposome complex of claim 20, wherein the liposome comprises phosphatidyl serine, phosphatidyl ethanolamine, and cholesterol in a 1:1:1 molar ratio.

22. The liposome complex of claim 1, further comprising a therapeutic payload.

23. The liposome complex of claim 22, wherein the therapeutic payload is a nucleic acid encoding a therapeutic protein, a drug, or a nucleic acid.

24. The liposome complex of claim 23, wherein the nucleic acid down-regulates expression of a cognate receptor for the peptide ligand.

25. The liposome complex of claim 24, wherein the nucleic acid is an antisense or siRNA molecule that specifically down-regulates mRNA encoding the cognate receptor.

26. The liposome complex of claim 23, wherein the nucleic acid is complexed with protamine.

27. The liposome complex of claim 23, wherein the therapeutic payload is a nucleic acid encoding a therapeutic protein.

28. The liposome according to claim 27, wherein the nucleic acid encoding the therapeutic protein comprises a plasmid.

29. The liposome according to claim 28, wherein the therapeutic protein is a cholinesterase.

30. The liposome according to claim 23, wherein the therapeutic payload is a therapeutic drug.

31. The liposome according to claim 23, wherein the therapeutic payload is an antisense oligonucleotide or siRNA.

32. A method of preparing a liposome complex of claim 1, comprising mixing the conjugated peptide ligand and lipids in an organic solvent, removing the organic solvent, and mixing said conjugated peptide ligand and lipids in an aqueous solution to form liposome complexes.

33. The method of claim 32, wherein the organic solvent is chloroform or chloroform:methanol.

34. The method of claim 32, further comprising sonicating the aqueous solution following said mixing.

35. The method of claim 32, wherein the aqueous solution comprises 8% detergent and wherein the method further comprises using reverse dialysis to extract the detergent.

36. The method of claim 32, wherein the liposome complex encapsulates a therapeutic payload.

37. The method of claim 36, wherein the therapeutic payload is a nucleic acid in a complex with protamine.

38. The method of claim 32, wherein said lipids are phosphatidyl serine, phosphatidyl ethanolamine, and cholesterol in a 1:1:1 molar ratio.

39. A method of preparing a liposome complex of claim 1, comprising:
(a) preparing a solution comprising: (i) PBS; (ii) a conjugated peptide ligand; and (iii) phospholipids and cholesterol;
(b) mixing the solution; and
(c) sonicating and extruding the solution through polycarbonate filters to form the liposome complex.

40. The method according to claim 39, wherein the phospholipids and cholesterol are phosphatidyl serine, phosphatidyl ethanolamine, and cholesterol in a 1:1:1 molar ratio.

41. A pharmaceutical composition comprising:
(a) a therapeutically effective amount of a liposome complex according to claim 1; and
(b) a pharmaceutically acceptable carrier.

42. A method of treating a receptor-associated disorder in a patient in need thereof, the method comprising administering to the patient a liposome complex according to claim 1 in an amount effective to treat the disorder, wherein the receptor-associated disorder comprises an over-abundance and/or up regulation of receptors.

43. The method of claim 42, wherein the receptor-associated disorder is an endothelin receptor-associated disease.

44. The method of claim 43, wherein the endothelin receptor-associated disease affects lungs or prostate.

45. A method of treating a receptor-associated disorder in a patient in need thereof, the method comprising administering to the patient a pharmaceutical composition according to claim 41 in an amount effective to treat the disorder, wherein the receptor-associated disorder comprises an over-abundance and/or up regulation of receptors.
